(19) United States
(12) Patent Application Publication
(10) Pub. No.: US 2007/0154996 A1
(43) Pub. Date: Jul. 5, 2007

(54) Method for transferring a glucosyl residue

(76) Inventors: Tomoyuki Nishimoto, Okayama (JP); Michio Kubota, Okayama (JP); Shigeharu Fukuda, Okayama (JP); Toshio Miyake, Okayama (JP)

Correspondence Address: BROWDY AND NEIMARK, P.L.L.C.;624 NINTH STREET, NW, SUITE 300, WASHINGTON, DC, 20001-5303, US

(21) Appl. No.: 10/587,711

(22) PCT Filed: Jan. 27, 2005
(86) PCT No.: PCT/JP05/01088

§ 371 Date: Jul. 28, 2006

(30) Foreign Application Priority Data
Jan. 28, 2004 (JP) 2004-019118

(51) Int. Cl. C12P 19/04

(52) U.S. Cl. 435/101

(57) ABSTRACT

An object of the present invention is to provide novel methods for forming glucosyl-transferred polyalcohols, glucosyl-transferred glucuronic acid, and glucosyl-transferred derivatives of glucose whose C-6 hydroxyl group bound to a saccharide by using an enzymatic reaction. The present invention solves the above object by providing a method for transferring a glucosyl residue to polyalcohols, glucuronic acid and/or derivatives of glucose whose C-6 hydroxyl group bound to a saccharide, comprising a step of: allowing a trehalose phosphorylase to act on a saccharide containing glucose as a component sugar and one or more polyalcohols selected from the group consisting of inositol, ribitol, erythritol, and glycerol; glucuronic acid and/or a salt thereof; and/or one or more derivatives of glucose whose C-6 hydroxyl group bound to a saccharide selected from the group consisting of isomaltose, gentiobiose, melibiose, isomaltotriose, and isopanose.

### TECHNICAL FIELD

The present invention relates to a novel method for transferring a glucosyl residue to a polyalcohol, glucuronic acid and/or a salt thereof (hereinafter, “glucuronic acid and/or a salt thereof” is simply abbreviated as “glucuronic acid” in this specification) and a derivative of glucose whose C-6 hydroxyl group bound to a saccharide (abbreviated as “C-6DG”, hereinafter), particularly, a novel method for transferring a glucosyl residue to a polyalcohol, glucuronic acid, and C-6DG by using a glucosyl-transferring activity of a trehalose phosphorylase.

### BACKGROUND ART

Recently, functions of various saccharides represented by oligosaccharides have been found one after another. Accordingly, demands for functional saccharides are diversified, and saccharides having more outstanding functions or those having extremely novel functions, for example, useful saccharides which can be used in a field except for foods, cosmetics, and pharmaceuticals, are required. In this art, researches for establishing novel methods for the purpose of producing novel or rare various saccharides on an industrial scale and those for estimating the functions of saccharides produced by the novel methods are in progress to meet the above demands.

Polyalcohols, a kind of saccharides (usually, called “sugar alcohols” or “polyols”), glucuronic acid, and C-6DGs have outstanding functions for edible materials having low cariogenisity, low digestivity, or salt-forming activity with minerals. Therefore, it is possible to provide saccharides having outstanding or novel functions by producing those related compounds such as glucosyl-transferred polyalcohols, glucosyl-transferred glucuronic acid, and glucosyl-transferred C-6DGs; and elucidating their functions.

As for methods for producing glucosyl-transferred polyalcohols, Japanese Patent Kokai Nos. 91,891/93, 65,293/2002, and 163,092/90 disclose the methods using the glucosyl-transferring activities of sucrose phosphorylase, kojibiose phosphorylase, and α-glucosidase. Also, as for method for producing glycosyl-transferred glucuronic acid, Japanese Patent Kokai No. 253,879/94 discloses the method for transferring a galactosyl residue from lactose by using β-galactosidase. Further, as for methods for producing glucosyl-transferred C-6DGs, various methods for transferring a glucosyl residue using sucrose phosphorylase, kojibiose phosphorylase, α-glucosidase, dextransucrease, cyclomaltodextrin glucanotransferase, etc., have been proposed. Products obtainable by those methods have different features such as saccharide compositions and structure of saccharides depending on the substrate specificities of enzymes. Therefore, it is considered that the products shall exhibit different functions. However, taking account of the diversified demands on saccharides in the present circumstance, the present inventors considered that the processes for producing glucosyl-transferred polyalcohols, glucosyl-transferred acidic saccharides, and glucosyl-transferred oligosaccharides, which had been proposed, were not sufficient to meet the demands, and concluded that various processes for producing those were required. Further, the present inventors considered that they can contribute to establish various processes for producing saccharides having more outstanding or novel functions by providing a method for forming glucosyl-transferred polyalcohols, glucosyl-transferred glucuronic acid, and glucosyl-transferred C-6DGs using a different enzyme from those used in conventional processes for producing glucosyl-transferred polyalcohols, glucosyl-transferred acidic saccharides, and glucosyl-transferred C-6DGs.

### DISCLOSURE OF INVENTION

Under these circumstances, an object of the present invention is to provide a novel method for forming glucosyl-transferred polyalcohols, glucosyl-transferred glucuronic acid, and glucosyl-transferred C-6DGs by using an enzyme.

To solve the above object, the inventors of the present invention firstly investigated on the activity of transferring a glucosyl residue of known saccharide-related enzymes, which were assumed to have a glycosyl-transferring activity to polyalcohols, using sorbitol, one of representative polyalcohols, as an acceptor. However, enzymes usable to solve the above object were not found by the above investigation. Successively, the inventors of the present invention widely screened enzymes having an activity of transferring a glucosyl residue to various polyalcohols except for sorbitol without regarding their glucosyl-transferring activity to polyalcohols. As a result, it was unexpectedly found that a trehalose phosphorylase, disclosed by the same applicant as the present invention of Japanese Patent Kokai No. 304,881/98, has an activity of transferring a glucosyl residue to polyalcohols including inositol. The result was unexpected from the description of the above specification that the enzyme showed no glucosyl-transferring activity on sorbitol. Based on the result, it had been considered to have no activity of transferring a glucosyl residue to polyalcohols.

The inventors of the present invention further investigated the glucosyl-transferring activity of trehalose phosphorylase using glucuronic acid, an acidic saccharide formed by the oxidation of C-6 position of glucose, and oligosaccharides which are derivatives of glucose derivatized at C-6 hydroxyl group with a glucosyl or other saccharide residues (C-6DGs) as acceptors. As a result, it was revealed that the trehalose phosphorylase has a significant activity of transferring a glucosyl residue to glucuronic acid and C-6DGs including isomaltose. Further, it was confirmed that the glucosyl-transferring reaction using the trehalose phosphorylase can be advantageously used for producing glucosyl-transferred polyalcohols, glucosyl-transferred glucuronic acid, and glucosyl-transferred C-6DGs on an industrial scale The present invention has been made based on the above original findings of the present inventors.

The present invention solves the above object by providing a method for transferring a glucosyl residue to polyalcohols, glucuronic acid and/or C-6DGs, comprising a step of:
allowing a trehalose phosphorylase to act on a saccharide containing glucose as a component sugar and one or more polyalcohols selected from the group consisting of inositol, ribitol, erythritol, and glycerol;
glucuronic acid and/or salts thereof; and/or
one or more C-6DGs selected from the group consisting of isomaltose, gentiobiose, melibiose, isomaltotriose, and isopanose.

The method of transferring a glucosyl residue of the present invention has features of high efficiency and forming lower by-products in comparison with conventional methods. The present invention enables the industrial production of glucosyl-transferred polyalcohols, glucosyl-transferred glucuronic acid, and glucosyl-transferred C-6DGs, which have been unknown or recognized as rare.

### BEST MODE FOR CARRYING OUT THE INVENTION

The method for transferring a glucosyl residue to polyalcohols, glucuronic acid, and C-6DG (hereinafter, may be called “the method of the present invention” or “the method” in this specification) of the present invention is characterized in that a trehalose phosphorylase is used for the transglucosylation. The word “trehalose” as referred to as in the present invention means a disaccharide represented by the formula, α-D-glucosyl α-D-glucoside. The word “trehalose phosphorylase” as referred to as in the present invention means an enzyme which catalyzes a reaction of phosphorolyzing a disaccharide, trehalose in the presence of inorganic phosphoric acid and/or its salt to form D-glucose and β-D-glucose 1-phosphoric acid and/or its salt (hereinafter, “β-D-glucose 1-phosphoric acid and/or its salt” is simply abbreviated as “β-G1P” in this specification) and vice versa. The trehalose phosphorylase usable in the present invention is defined as above, and not restricted by its origin and preparation method as far as it transfers a glucosyl residue to one or more polyalcohols, glucuronic acid, and/or one or more C-6DGs. For example, either of a natural enzyme from Thermoanaerobium brockii (ATCC 35047) and the recombinant enzyme, disclosed in Japanese Patent Kokai No. 304,881/98 applied for by the same applicant as the present invention, can be advantageously used. Also, the mutated enzyme, obtainable by applying methods of protein engineering to a DNA encoding the enzyme, disclosed in the application, can be used in the present invention as far as the mutated enzyme does not lose the objective transferring activity. Further, trehalose phosphorylases from other microorganisms, for example, trehalose phosphorylase from a microorganism of the genus Plesiomonas, disclosed in Japanese Patent Kokai No. 131,157/96, can be advantageously used as far as they catalyze the objective transferring reaction.

The word “polyalcohol” as referred to as in the present invention means an alcohol bearing two or more hydroxyl groups in the molecule and also means a compound usually called to “polyol” or “sugar alcohol”. A polyalcohol usable in the present invention as an acceptor of a glucosyl residue is one or more polyalcohols selected from the group consisting of inositol, ribitol, erythritol, and glycerol and is not restricted by the preparation method and its existence form. For example, polyalcohol preparations isolated from natural sources, including commercially available products; enzymatically prepared products or chemically synthesized products; polyalcohol preparations containing other concomitants, not affecting on the enzyme reaction in the present invention or use of the transfer products; and compositions containing the above preparations can be used in the present invention. Several stereoisomers such as myo-inositol, D-inositol, and L-inositol are present as inositol. These stereoisomers of inositol can be advantageously used in the present invention. Among them, myo-inositol is particularly useful in the present invention because it gives relatively large amount of glucosyl-transferred product.

The word “glucuronic acid” as referred to as in the present invention means an acidic saccharide having a structure where C-6 position of D-glucose is oxidized to carboxyl group and is not restricted by the preparation method and its existence form. Glucuronic acid preparations isolated from natural sources, including commercially available products; enzymatically prepared products or chemically synthesized products; glucuronic acid preparations containing other concomitants, not affecting on the enzyme reaction in the present invention or use of the transfer products; and compositions containing the above preparations can be used in the present invention.

The word “C-6 DG” as referred to as in the present invention means a derivative of glucose whose C-6 hydroxyl group is bound to other saccharide. The C-6DG usable in the present invention as an acceptor of a glucosyl residue is one or more saccharides selected from the group consisting of isomaltose, gentiobiose, melibiose, isomaltotriose, and isopanose and is not restricted to specific preparation method and its existence form. For example, C-6DG preparations isolated from natural sources, including commercially available products; enzymatically prepared products or chemically synthesized products; C-6DG preparations containing other concomitants, not affecting on the enzyme reaction in the present invention or use of the transfer products; and compositions containing any of the above preparations can be used in the present invention.

A saccharide containing glucose as a component sugar, usable in the present invention, means a derivative of glucose or oligosaccharide, containing glucose as a component sugar, used as a glucosyl donor in the glucosyl-transferring reaction by trehalose phosphorylase. Such saccharide is not restricted to specific preparation method and its existence form. For example, the saccharide preparations isolated from natural sources, including commercially available products; enzymatically prepared products or chemically synthesized products; the saccharide preparations containing other concomitants, not affecting on the enzyme reaction in the present invention or use of the transfer products; and compositions containing any of the above preparations can be used in the present invention. As such saccharide, β-G1P is relatively preferable. β-G1P can be prepared enzymatically by the steps of:
allowing a trehalose phosphorylase to act on trehalose,
allowing a maltose phosphorylase (a product commercialized by Oriental Yeast Co.) to act on maltose, or
allowing kojibiose phosphorylase (disclosed in Japanese Patent Kokai No. 304,882/98 applied for by the same applicant as the present invention) to act on koji-oligosaccharides, having a structure of binding glucoses via α-1,2 glucosidic linkages, such as kojibiose and kojitriose, to form β-G1P; and
purifying the resulting β-G1P to the objective level.

Also, oligosaccharides containing glucose as a component sugar, which releases β-G1P by an enzyme action, can be used intact in the present invention. In the case of using trehalose as such an oligosaccharide, the formation of β-GLP and transglucosylation to polyalcohols, glucuronic acid, or C-6DGs by trehalose phosphorylase proceed simultaneously. In the case of using maltose and/or koji-oligosaccharides, the objective transglucosylation can be carried out by allowing the above maltose phosphorylase and/or kojibiose phosphorylase to act on the saccharides together with trehalose phosphorylase.

A glucosyl residue can be transferred to polyalcohols, glucuronic acid, and C-6DGs by trehalose phosphorylase by adding trehalose phosphorylase to an aqueous solution containing polyalcohols, glucuronic acid, and/or C-6DGs and saccharides containing glucose as a component sugar (hereinafter, one or more of the polyalcohols, glucuronic acid, C-6DGs and saccharides containing glucose as a component sugar may be called “substrate(s)”); and keeping the resulting mixture under the condition adequately selected according to the enzymatic properties of trehalose phosphorylase. In the case of using a trehalose phosphorylase disclosed in Japanese Patent Kokai No. 304,881/98 applied for by the same applicant as the present invention, the condition for the reaction can be selected as far as the trehalose phosphorylase does not lose its activity. The reaction temperature can be set to, usually, 70° C. or lower, more preferably, 65° C. or lower. The reaction pH can be set to, usually, pH 4.0 to 9.0, more preferably, pH 5.0 to 7.5. The substrate concentration in the reaction mixture is not restricted as far as the objective reaction can proceed. For example, the concentration of polyalcohols and saccharides containing glucose as a component sugar is preferably set to, usually, 0.1 to 40% (w/w), desirably, 0.2 to 20% (w/w). The ratio of those is preferably set to, usually, 1:0.1 to 400, desirably, 1:1 to 100, more desirably, 1:2 to 50. In the case of using trehalose, maltose and/or kojibiose as a saccharide containing glucose as a component sugar and using maltose phosphorylase and/or kojibiose phosphorylase, it is preferable that inorganic phosphoric acid and/or its salt, for example, sodium dihydrogen phosphate is added to the reaction mixture to give an adequate concentration, usually, 0.5 to 100 mM, desirably, 1 to 50 mM. In the case of using maltose phosphorylase and/or kojibiose phosphorylase together with trehalose phosphorylase, it is preferable to select the condition where all enzymes are not inactivated, in consideration of enzymatic properties of the enzymes. The preferable amount of trehalose phosphorylase is, usually, 0.1 to 500 units, desirably, 0.5 to 200 units to one gram of the total amount, on a dry solid basis, of substrates in the reaction mixture. One unit of trehalose phosphorylase as referred to as in the present invention is defined as the amount of enzyme which forms one μmol of D-glucose per one minute from trehalose under the conditions at pH 5.5 and 60° C. according to the method described in Japanese Patent Kokai No. 304,881/98 applied for by the same applicant as the present invention. In the case of using the above reaction mixture, the reaction time can be properly selected according to the progress of the reaction, and set to, usually, 2 to 200 hours, desirably, 4 to 100 hours.

Usually, the reaction temperature is preferably set to a higher level as much as possible in the case of producing saccharides enzymatically. By using a higher reaction temperature, contamination of the reaction mixture can be prevented, the reaction can be accelerated, and a higher substrate concentration can be used. As a result, the objective reaction can be proceeded more efficiently. Similarly, in the case of the transglucosylation of the present invention, the reaction temperature is preferably set to, usually, ambient temperature or higher, desirably, 40° C. or higher, more desirably, 50° C. or higher. Therefore, it is preferable to use a trehalose phosphorylase having a good thermal stability, for example, the enzyme which keeps, usually, 80% or higher, desirably, 85% or higher, more desirably, 90% or higher of the inherent activity when incubated at 60° C. for one hour under its optimum pH condition. A trehalose phosphorylase disclosed in Japanese Patent Kokai No. 304,881/98 applied for by the same applicant as the present invention can be advantageously used to the present invention because the enzyme has a good thermal stability as described above.

By the method of transferring a glucosyl residue of the present invention, glucosyl-transferred polyalcohols, glucosyl-transferred glucuronic acid, and/or glucosyl-transferred C-6DGs are formed in the reaction mixture. The word, “glucosyl-transferred polyalcohol” as referred to as in the present invention means a saccharide having a structure of binding a polyalcohol and a glucosyl residue via a covalent bond. Also, the word, “glucosyl-transferred glucuronic acid” as referred to as the present invention means a saccharide having a structure of binding a glucuronic acid and a glucosyl residue via a covalent bond. The word, “glucosyl-transferred C-6DG” as referred to as in the present invention means a saccharide having a structure of binding a C-6DG and a glucosyl residue via a covalent bond. A glucosyl-transferred polyalcohol formed by the method of the present invention has a polyalcohol and a glucosyl residue as component units. A glucosyl-transferred glucuronic acid formed by the method of the present invention has a glucuronic acid and a glucosyl residue as component units. When a C-6DG has no glucosyl residue in its molecule, the glucosyl-transferred C-6DG formed by the method of the present invention has a glucosyl residue. When a C-6DG has one or more glucosyl residues in its molecule, the glucosyl-transferred C-6DG of the present invention has a glucosyl residue except for glucosyl residues of the C-6DG. The linkage of binding component units of a glucosyl-transferred polyalcohol, glucosyl-transferred glucuronic acid, and glucosyl-transferred C-6DG, formed by the method of the present invention, may comprise a linkage specific to the present invention, which is hardly formed by the method for transferring a glucosyl residue using enzymes except for trhalose phosphorylase. For example, in the case of using inositol, a cyclic polyalcohol with six carbon atoms, as an acceptor, the resulting glucosyl-transferred polyalcohol may have α-1,1′ glucosidic linkage as a linkage of binding component units.

Glucosyl-transferred polyalcohols, glucosyl-transferred glucuronic acid and/or glucosyl-transferred C-6DGs, formed by the method of the present invention, can be used for various uses in the form of an intact reaction mixture or preparation purified to an objective level by conventional methods. Therefore, the method of the present invention can be advantageously used as a step of producing glucosyl-transferred polyalcohols, glucosyl-transferred glucuronic acid, glucosyl-transferred C-6DG and/or compositions comprising those glucosyl-transferred products. The present invention also provides a process for producing glucosyl-transferred polyalcohols, glucosyl-transferred glucuronic acid, glucosyl-transferred C-6DG and/or compositions comprising those glucosyl-transferred products. The process comprising the steps of: (a) transferring a glucosyl-residue by the method of the present invention; and (b) collecting glucosyl-transferred polyalcohols, glucosyl-transferred glucuronic acid, glucosyl-transferred C-6DG and/or compositions comprising those glucosyl-transferred products, formed in the step (a). Glucosyl-transferred polyalcohols, glucosyl-transferred glucuronic acid, glucosyl-transferred C-6DG and/or compositions comprising those glucosyl-transferred products, formed by the method of the present invention can be collected by the adequate conventional methods, for example, one or more methods selected from the group consisting of decoloration using active charcoal, deionization using ion-exchange resins, filtration using diatom earth as an auxiliary agent, chromatography using ion-exchange resins, concentration using a evaporator, drying such as spray-drying, drying in vacuo, and freeze-drying, and crystallization using a adequate solvent such as water and alcohols. The products obtainable by the above process of the present invention are provided in suitable forms such as powder, crystalline powder, granule, block, syrup, etc.; comprising glucosyl-transferred polyalcohols, glucosyl-transferred glucuronic acid, glucosyl-transferred C-6DG in various purities such as highly purified crystalline or composition comprising other components. As in the case of polyalcohols, glucuronic acid, or C-6DGs used as acceptors in the present invention, the products obtainable by the process of the present invention can be advantageously used in various fields such as foods including health foods and beverages, cosmetics, pharmaceuticals, feeds, etc., as a sweetener, hardly digestive sweetener, low cariogenic sweetener, moisture-retaining agent, starch-retrogradation preventing agent, antiflatulent, mineral-adsorption promoting agent, etc. Also, the products obtainable by the method of the present invention can be used as investigative reagents for estimating their functions. Based on the investigative results, glucosyl-transferred polyalcohols, glucosyl-transferred glucuronic acid, glucosyl-transferred C-6DG and/or compositions comprising those glucosyl-transferred products, obtainable by the process of the present invention can be used in the above fields as ingredients of various functional agents such as antiseptic agent, preservative, antimicrobial agent, antiviral agent, vital function-controlling agent, etc.

The following examples explain the present invention in detail.

### EXAMPLE 1

### Transglucosylation to Polyalcohols

### Example 1-1

### Preparation of a Trehalose Phosphorylase

According to the method disclosed in Japanese Patent Kokai No. 304,881/98, applied for by the same applicant as the present invention, Thermoanaerobium brockii (ATCC 35047) was cultivated in a culture medium containing trehalose as a carbon source on a 40-liters culture scale. Successively, according to the method described in the above application, cells collected from the culture were disrupted by ultrasonication and then, the supernatant was collected. The trehalose phosphorylase activity of the supernatant detected by the assay method of trehalose phosphorylase activity described in the above application.

By concentrating the above supernatant with a UF-membrane, 360 ml of an enzyme solution having a trehalose phosphorylase activity of about 30 units/ml was obtained. According to the method described in the above application, 300 ml of the enzyme solution was subjected to ion-exchange chromatography using “DEAE-TOYOPEARL”, a gel commercialized by Tosoh Corporation, Tokyo, Japan, hydrophobic chromatography using “BUTYL-TOYOPEARL 650”, a gel commercialized by Tosoh Corporation, Tokyo, Japan, and gel filtration chromatography using “ULTROGEL AcA44”, a gel commercialized by Sepracor, France, to obtain a purified trehalose phosphorylase preparation showing a single band on 7.5%(w/v) polyacrylamide gel electrophoresis. The specific activity of the purified enzyme preparation thus obtained was about 78 units/mg-protein.

### Example 1-2

### Transglucosylation by the Action of Trehalose Phosphorylase

An aqueous solution containing 1% (w/v) of either of polyalcohols (all reagent grade), shown in Table 1 below, 1.4% (w/v) of a reagent grade β-G1P, one unit/ml of trehalose phosphorylase obtained in Example 1-1, and 50 mM acetate buffer (pH 6.0) was prepared and followed by the enzyme reaction while keeping the solution at 50° C. for 24 hours. After the reaction, a portion of each reaction mixture was withdrawn, dried, and dissolved in pyridine for gas chromatography analysis (hereinafter, simply abbreviated as “GC”). In GC analysis, a stainless steel column (internal diameter 3 mm×length 2 m) packed with 2% Silicon OV-17/Chromosorb W”, commercialized by GL Science, Tokyo, Japan) was used. Nitrogen gas was used as carrier gas and the flow rate was set to 40 ml/min. A column oven was controlled to rise a column temperature at 160° C. to 320° C. in a rate of 7.5° C./min after the injection of samples. A hydrogen-flame ion detector was used for the detection. An aqueous solution with the same composition except for trehalose phosphorylase was prepared, kept with the same condition described above, and analyzed by GC by the same condition to confirm the chromatogram of non-reacted polyalcohol and β-G1P. Transfer of a glucosyl residue to each polyalcohol was judged by detecting new peak(s) in the chromatogram of the reaction mixture. The amount of glucosyl-transferred polyalcohol formed by the reaction was estimated based on a peak area of the glucosyl-transferred polyalcohol to the total peak area including that of non-reacted (residual) polyalcohol, and classified into three groups of the area of 60% or higher, “+++”; 30% or higher but less than 60%, “++”; and 0% or higher but less than 30%, “+”. These results were shown in Table 1 together with retention times in GC of the formed glucosyl-transferred polyalcohols.

TABLE 1

| | Transfer of | Retention time of |
|---|---|---|
| | a glucosyl residue | transferred product |
| Polyalcohol | (Transglucosylation) | (min) |
| Sorbitol | −* | N.D. |
| myo-Inositol | ++ | 15.4 and 16.6 |
| Erythritol | + | 11.5 |
| Ribitol | + | 13.6 |
| Glycerol | + | 9.6 |

*Transglucosylation was not detected.

As shown in Table 1 and as described in Japanese Patent Kokai No. 304,881/98 applied for by the same applicant as the present invention, trehalose phosphorylase originated from Thermoanaerobium brockii, prepared in Example 1-1, showed no transglucosylation on sorbitol. In contrast, it was confirmed that the trehalose phosphorylase transferred a glucosyl residue to other polyalcohols such as myo-inositol, erythritol, ribitol, and glycerol. Among the polyalcohols, a glucosyl residue was remarkably transferred to myo-inositol judged to “++”. In the case of myo-inositol, it was found that two kinds of glucosyl-transferred myo-inositol were formed, because two peaks of glucosyl-transferred products (Rt: 15.4 and 16.6 min) were detected by GC analysis.

The two glucosyl-transferred polyalcohols were purified from respective reaction mixtures to the level of substantially showing a single peak and no other peak of by-product by GC analysis by using a conventional method including preparative HPLC using an ion-exchange resin. According to the method described in Doudroff et al., “THE ENZYMES” Vol. 5, pp. 229-236, published by Academic Press (1961), the purified preparations were phosphorolyzed by trehalose phosphorylase in the presence of arsenic trioxide and the products were analyzed by GC. In both chromatograms, peaks corresponding to a polyalcohol and D-glucose were detected in a molar ratio of 1:1, calculated from respective peak areas. The results mean that the purified preparations are glucosyl-transferred polyalcohols where a polyalcohol and a glucosyl residue are bound together in a molar ratio of 1:1. Two kinds of glucosyl-transferred polyalcohols formed from myo-inositol were compounds of binding myo-inositol and glucosyl residue in a molar ratio of 1:1. These results indicate that two kinds of glucosyl-transferred polyalcohol with different linkages were formed by the transglucosylation to myo-inositol.

### Example 1-3

### Transglucosylation to Glucuronic Acid and C-6DGs by the Action of Trehalose Phosphorylase

An aqueous solution containing 1% (w/v) of any one of glucuronic acid or C-6DGs (all reagent grade), shown in Table 2 below, 1.4% (w/v) of a reagent grade β-G1P, one unit/ml of trehalose phosphorylase obtained in Example 1-1, and 50 mM acetate buffer (pH 6.0) was prepared and followed by the enzyme reaction while keeping the solution at 50° C. for 24 hours. After the reaction, each reaction mixture was analyzed by GC described in Example 1-2. In the same manner as described in Example 1-2, the amount of a glucosyl-transferred product formed by the reaction was estimated based on a peak area of the glucosyl-transferred product to the total peak area including that of non-reacted (remaining) glucuronic acid or C-6DGs, and classified into three groups of the area of 60% or higher, “+++”; 30% or higher but less than 60%, “++”; and 0% or higher but less than 30%, “+”. These results were shown in Table 2.

TABLE 2

| | Transfer of | Retention time of |
|---|---|---|
| Glucuronic acid or | a glucosyl residue | transferred product |
| C-6DGs | (Transglucosylation) | (min) |
| Glucuronic acid | + | 15.6 |
| Isomaltose | +++ | 22.1 |
| Gentiobiose | +++ | 22.1 |
| Melibiose | +++ | 21.9 |
| Isomaltotriose | +++ | 31.6 |
| Isopanose | +++ | 29.7 |

As shown in Table 2, trehalose phosphorylase transferred a glucosyl residue to all acceptors, glucuronic acid and C-6DGs such as isomaltose, gentiobiose, melibiose, isomaltotriose, and isopanose. Particularly, in the case of isomaltose, gentiobiose, melibiose, isomaltotriose, and isopanose, which are C-6DGs bearing a glucose at the non-reducing end and the C-6 hydroxyl group of the glucose being linked with other saccharides, the transglucosylation was remarkable judged to “+++” in comparison with the case of glucuronic acid judged to “+”. In addition, it was revealed that the method of transferring a glucosyl residue of the present invention has a merit of hardly forming by-products, because substantially one kind of glucosyl-transferred C-6DG was formed by trehalose phosphorylase in any case of the above C-6DGs.

The glucosyl-transferred glucuronic acid and glucosyl-transferred C-6DG were purified from respective reaction mixtures to a level of substantially showing a single peak by GC analysis by using a conventional method including preparative HPLC using an octadecyl silica gel. The purified preparations were respectively phosphorolyzed by trehalose phosphorylase in the presence of arsenic trioxide and the products were analyzed by GC. In any chromatogram, peaks corresponding to glucuronic acid or a C-6DG and D-glucose were detected in a molar ratio of 1:1, calculated from respective peak areas. It was also revealed that all products show no reducing power by measuring the reducing power of the purified preparations by Somogyi-Nelson method. These results mean that the purified preparations are glucosyl-transferred glucuronic acid or glucosyl-transferred C-6DGs where glucuronic acid or C-6DGs and a glucosyl residue are bound together in a molar ratio of 1:1. Also, the results mean that glucuronic acid was bound to D-glucose at C-1 position, and that isomaltose, gentiobiose, melibiose, isomaltotriose and isopanose were bound to D-glucose at those C-1 positions of glucose at the reducing ends.

### Example 2

### Production of Syrup Comprising Glucosyl-Transferred myo-inositol

An aqueous solution containing 2% (w/v) of β-G1P, 10%(w/v) of myo-inositol, and one unit/ml of trehalose phosphorylase obtained by the method in Example 1-1 was adjusted to pH 6.0 and kept at 60° C. for 72 hours for transferring a glucosyl residue to myo-inositol. Then, the resulting reaction mixture was decolored and deionized by conventional methods, and fractionated by a column chromatography using an ion-exchange resin. A portion of the resulting each fraction was analyzed by the conventional method, and fractions showing a relatively high content, on a dry solid basis, of glucosyl-transferred myo-inositol, in comparison with the content of reaction mixture, were mixed. The resulting solution was concentrated to give about 72%, on a dry solid basis, syrup comprising glucosyl-transferred myo-inositol. A portion of the resulting syrup was analyzed by GC described in Example 1-2, and the content, on a dry solid basis, of glucosyl-transferred myo-inositol in the syrup was calculated based on a peak area of the GC chromatogram. As a result, the content was estimated to be about 60%.

The product can be advantageously used in various fields such as foods and beverages including healthy foods and beverages, feeds and baits, cosmetics, and pharmaceuticals, as a sweetener, low-digestive sweetener, low cariogenic sweetener, moisture-retaining agent, starch-retrogradation preventing agent, antiflatulent, etc.

### Example 3

Production of Syrup Comprising Glucosyl-Transferred Glucuronic Acid

An aqueous solution containing 20% (w/v) of trehalose, 10 mM of dipotassium phosphate-citric acid buffer (pH 6.0), 2% (w/v) of sodium glucuronate, and 20 units/ml of trehalose phosphorylase obtained by the method in Example 1-1 was prepared and kept at 55° C. for 96 hours for transferring a glucosyl residue to glucuronic acid. Then, the resulting reaction mixture was decolored by conventional methods, and adsorbed on an ion-exchange resin. The adsorbent was eluted with diluted hydrochloric acid. The resulting solution was fractionated by a column chromatography using an ion-exchange resin. A portion of the resulting each fraction was analyzed by conventional method, and fractions containing glucosyl-transferred glucuronic acid were mixed. The resulting solution was neutralized and concentrated to give about 60%, on a dry solid basis, syrup comprising glucosyl-transferred glucuronic acid. A portion of the resulting syrup was analyzed by GC described in Example 1-2, and the content, on a dry solid basis, of glucosyl-transferred glucuronic acid in the syrup was calculated based on a peak area of the GC chromatogram. As a result, the content was estimated to be about 70%.

The product can be advantageously used in various fields such as foods and beverages including health foods and beverages, feeds, cosmetics, and pharmaceuticals, as an acidifier, sweetener, moisture-retaining agent, mineral-stabilizing agent, etc.

### Example 4

### Production of Syrup Comprising Glucosyl-Transferred Isomaltose

An aqueous solution containing 20% (w/v) of trehalose, 5 mM of dipotassium phosphate-citric acid buffer (pH 6.0), 20% (w/v) of isomaltose, and 10 units/ml of trehalose phosphorylase obtained by the method in Example 1-1 was prepared and kept at 60° C. for 72 hours for transferring a glucosyl residue to isomaltose. Then, the resulting reaction mixture was decolored and deionized by conventional methods, and fractionated by a column chromatography using an ion-exchange resin. A portion of the resulting each fraction was analyzed by conventional method, and fractions showing a relatively high content, on a dry solid basis, of glucosyl-transferred isomaltose, in comparison with the content of reaction mixture, were mixed. The resulting solution was concentrated to give about 75%, on a dry solid basis, syrup comprising glucosyl-transferred isomaltose. A portion of the resulting syrup was analyzed by GC described in Example 1-2, and the content, on a dry solid basis, of glucosyl-transferred isomaltose in the syrup was calculated based on a peak area of the GC chromatogram. As a result, the content was estimated to be about 50%.

The product can be advantageously used in various fields such as foods and beverages including healthy foods and beverages, cosmetics, pharmaceuticals, and feeds as a sweetener, low-digestive sweetener, low cariogenic sweetener, moisture-retaining agent, starch-retrogradation preventing agent, antiflatulent, etc.

### INDUSTRIAL APPLICABILITY

As described above, the present invention was established based on an original knowledge of the present inventors that a trehalose phosphorylase catalyzes a reaction of transferring a glucosyl residue to inositol, ribitol, erythritol, glycerol, glucuronic acid, isomaltose, gentiobiose, melibiose, isomaltotriose, and isopanose. By using the method of the present invention, glucosyl-transferred polyalcohols, glucosyl-transferred glucuronic acid, and glucosyl-transferred C-6DGs, which has been unknown or recognized as rare sugars, can be produced on an industrial scale. A composition comprising the glucosyl-transferred polyalcohol, glucosyl-transferred glucuronic acid, and/or glucosyl-transferred C-6DGs, which is produced by the method of the present invention, can be advantageously used in various fields such as foods and beverages including healthy foods and beverages, feeds and baits, cosmetics, pharmaceuticals, and reagents for research works. The present invention, having these outstanding functions and effects, is a significantly important invention. that greatly contributes to this art.

1. A method for transferring a glucosyl residue to a polyalcohol, comprising a step of:
allowing a trehalose phosphorylase to act on a saccharide containing glucose as a component sugar, and one or more polyalcohols selected from the group consisting inositol, ribitol, erythritol, and glycerol.

2. A method for transferring a glucosyl residue to glucuronic acid and/or a salt thereof, comprising a step of:
allowing a trehalose phosphorylase to act on a saccharide containing glucose as a component sugar, and glucuronic acid and/or salts thereof.

3. A method for transferring a glucosyl residue to a derivative of glucose whose C-6 hydroxyl group bound to a saccharide:
allowing a trehalose phosphorylase to act on a saccharide containing glucose as a component sugar, and one or more derivatives of glucose whose C-6 hydroxyl group bound to a saccharide, selected from the group consisting isomaltose, gentiobiose, melibiose, isomaltotriose and is isopanose.

4. The method of any one of claims 1 to 3, wherein said saccharide containing glucose as a component sugar is β-D-glucose-1-phosphate and/or a salt thereof or trehalose.

5. The method of any one of claims 1 to 3, wherein said trehalose phosphorylase has a thermal stability of keeping 80% or higher phosphorolytic activity when the enzyme is treated at pH 7.0 and 60° C. for one hour.

6. The method of any one of claims 1 to 3, wherein said trehalose phosphorylase is a natural enzyme originated from Thermoanaerobium brockii or a recombinant enzyme thereof.

7. A process for producing a glucosyl-transferred polyalcohol or a composition comprising the same, comprising the steps of:
forming the glucosyl-transferred polyalcohol by the method of claim 1; and
collecting the formed glucosyl-transferred polyalcohol or the composition comprising the same.

8. The process of claim 7, wherein the formed glucosyl-transferred polyalcohol or a composition comprising the same is collected by one or more methods selected from the group consisting of decoloring, deionization, filtration, concentration, chromatography, drying and crystallization.

9. A process for producing a glucosyl-transferred glucuronic acid and/or a salt thereof or a composition comprising the same, comprising the steps of:
forming the glucosyl-transferred glucuronic acid and/or a salt thereof by the method of claim 2; and
collecting the formed glucosyl-transferred glucuronic acid and/or a salt thereof or the composition comprising the same.

10. The process of claim 9, where the formed glucosyl-transferred glucuronic acid and/or a salt thereof or a composition comprising the same is collected by one or more methods selected from the group consisting of decoloring, deionization, filtration, adsorption, ion dialysis, concentration, chromatography, drying and crystallization.

11. A process for producing a glucosyl-transferred derivative of glucose whose C-6 hydroxyl group bound to a saccharide or a composition comprising the same, comprising the steps of:
forming the glucosyl-transferred derivative of glucose whose C-6 hydroxyl group bound to a saccharide by the method of claim 3; and
collecting the formed glucosyl-transferred derivative of glucose whose C-6 hydroxyl group bound to a saccharide or the composition comprising the same.

12. The process of claim 11, wherein the formed glucosyl-transferred derivative of glucose whose C-6 hydroxyl group bound to a saccharide or a composition comprising the same is collected by one or more methods selected from the group consisting of decoloring, deionization, filtration, adsorption, ion dialysis, concentration, chromatography, drying and crystallization.
